(19) United States
(12) Patent Application Publication
(10) Pub. No.: US 2003/0224011 A1
(43) Pub. Date: Dec. 4, 2003

(54) Hepatitis c virus conjugates

(76) Inventors: Anthony J. Conley, Exton, PA (US); Philip M. McKenna, Ardsley, PA (US); Craig T. Przysiecki, Lansdale, PA (US); Paul M. Keller, Lansdale, PA (US)

Correspondence Address: MERCK AND CO INC, P O BOX 2000, RAHWAY, NJ, 070650907

(21) Appl. No.: 10/296,353

(22) PCT Filed: May 29, 2001
(86) PCT No.: PCT/US01/17302

(51) Int. Cl. C12Q 1/70; A61K 39/29; C07K 14/02

(52) U.S. Cl. 424/189.1; 530/350; 435/5

(57) ABSTRACT

The present invention features HCV conjugates able to induce an immune response recognizing different strains and variants of HCV. The conjugates contain a polypeptide or protein complex carrier and one or more HCV mimotopes. Preferred HCV mimotopes provide antigens able to generate antibodies recognizing the hypervariable region of the HCV E2 protein.

### CROSS-REFERENCE TO RELATED APPLICATIONS

The present application claims priority to provisional application U.S. Serial No. 60/209,089, filed Jun. 2, 2000, hereby incorporated by reference herein.

### BACKGROUND OF THE INVENTION

The hepatitis C virus (HCV) is a major cause of acute, active, or persistent non-A and non-B viral hepatitis. HCV has a high mutation rate leading to the production of variants that may escape immune responses directed to the initial infection. (Puntoriero, et al., EMBO Journal 17:3521-3533, 1998.) HCV infection often causes persistent infection and frequently leads to cirrhosis and hepatocellular carcinoma. (See, Alter, Blood 85:1681-1695, 1995.)

Antibodies recognizing HCV can be elicited using both recombinant HCV protein and artificial peptides. Recombinant HCV protein used to elicit antibodies recognizing HCV include structural proteins E1 and E2. (Choo, et al., Proc. Natl. Acad. Sci. USA, 91:1294-1298, 1994.) Artificial peptides having amino acid sequences different from that found in HCV and yet able to generate antibodies recognizing HCV protein have been selected using phage display libraries. (Prezzi, et al., The Journal of Immunology 156:4504-4513, 1996; and Puntoriero, et al. EMBO Journal 17:3521-3533, 1998.) Such artificial peptides are referred to as HCV mimotopes.

### SUMMARY OF THE INVENTION

The present invention features HCV conjugates able to induce an immune response recognizing different strains and variants of HCV. The conjugates contain a polypeptide or protein complex carrier and one or more HCV mimotopes. Preferred HCV mimotopes provide antigens able to generate antibodies recognizing the hypervariable region of the HCV E2 protein.

Thus, a first aspect of the present invention describes an HCV conjugate. The conjugate comprises a polypeptide or protein complex carrier and an immunogenic HCV peptide, or a pharmaceutically acceptable salt thereof. The HCV peptide is covalently joined to the carrier. Additional groups such as other types of HCV peptides, reactive site capping groups, and anions may also be attached to the carrier.

“Covalently joined” refers to the presence of a covalent linker that is stable to hydrolysis under physiological conditions. Preferably, the covalent linker is stable to other reactions that may occur under physiological conditions including adduct formation, oxidation, and reduction.

In an embodiment of the present invention, covalently joined is achieved by “means for joining”. Such means cover the corresponding structure, material, or acts described herein and equivalents thereof.

An immunogenic HCV peptide comprises one or more antigenic determinants that can elicit an immune response recognizing HCV. Preferred antigenic determinants are HCV mimotopes that can elicit antibodies recognizing different strains and variants of HCV. Examples of HCV peptides are those comprising the HCV mimotope sequence of SEQ. ID. NO. 1, SEQ. ID. NO. 2, SEQ. ID. NO. 3, SEQ. ID. NO. 4, SEQ. ID. NO. 5, SEQ. ID. NO. 6, or SEQ. ID. NO. 7.

Preferably, the HCV conjugate comprises: a polypeptide or protein complex carrier, immunogenic HCV peptide PEP₁, and immunogenic HCV peptide PEP₂, wherein PEP₁ and PEP₂ are each covalently joined to the carrier though an independently selected covalent linker and comprises a different sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7; or a pharmaceutically acceptable salt thereof. In addition to two peptides comprising a sequence of SEQ. ID. NO. 1-7, the HCV conjugate may contain additional peptides including, but not limited, to peptides of SEQ. ID. NOs. 1-7.

Another aspect of the present invention describes an HCV conjugate produced by a process comprising: (a) joining a plurality of linkers to reactive sites on a polypeptide or protein complex carrier; (b) joining two or more different HCV immunogenic peptides to the plurality of linkers; and (c) capping the product of step (b). Capping is achieved by attaching a capping group to linkers not reacted with immunogenic peptides. Each of the two or more different HCV immunogenic peptides comprises either SEQ. ID. NO. 1, SEQ. ID. NO. 2, SEQ. ID. NO. 3, SEQ. ID. NO. 4, SEQ. ID. NO. 5, SEQ. ID. NO. 6, or SEQ. ID. NO. 7.

Reactive sites are groups capable of conjugation. Preferred reactive sites are nucleophilic sites, such as the epsilon amino group of lysine. A plurality of linkers refers to more than one linker.

The process can contain additional steps such as joining one or more anions and joining additional HCV immunogenic peptides other than those comprising SEQ. ID. NOs. 1-7. Such additional HCV immunogenic peptides are in addition to the two or more different HCV immunogenic peptides of SEQ. ID. NOs. 1-7.

Another aspect of the present invention describes an HCV conjugate mixture comprising a first and a second different HCV conjugate described herein. Preferably, the first HCV conjugate comprises a polypeptide or protein complex carrier covalently joined to a first immunogenic HCV peptide comprising SEQ. ID. NO. 1, to a second immunogenic HCV peptide comprising SEQ. ID. NO. 2 and to a third immunogenic HCV peptide comprising SEQ. ID. NO. 4, or a pharmaceutically acceptable salt thereof; and the second conjugate comprises an immunogenic HCV peptide comprising SEQ. ID. NO. 7, or a pharmaceutically acceptable salt thereof. The first and second HCV conjugates may contain additional groups attached to the respective carriers such as additional types of HCV peptides, reactive site capping groups, and anions.

Another aspect of the present invention describes a method of inducing an immune response in a subject. The method comprises the step of inoculating the subject with an effective amount of an HCV conjugate described herein.

Another aspect of the present invention describes a method of making an HCV conjugate containing multiple immunogenic HCV peptides. The method comprises the step of simultaneously conjugating the peptides to a carrier.

Another aspect of the present invention describes an antisera containing antibodies recognizing HCV. The antisera is made by a process comprising the steps of: (a) inoculating a subject with an effective amount of an HCV conjugate described herein to produce antibodies; and (b) removing the antibodies from the subject.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### DETAILED DESCRIPTION OF THE INVENTION

The present invention features HCV conjugates containing a polypeptide or protein complex carrier and one or more HCV mimotopes. The conjugates are able to elicit antibodies recognizing the HCV mimotope used to induce the immune response and also recognizing different strains and variants of HCV.

Different techniques can be employed to use an antigen to elicit an immune response. The examples provided below include data illustrating benefits to using an immunogenic protein carrier complex and to using particular antigens or antigen combinations. These results suggest that an HCV mimotope delivered to a subject as part of a conjugate containing immunogenic polypeptide or protein complexes have advantages in eliciting an immune response compared to the same HCV mimotope peptide delivered to the subject using other techniques such as chimeric mimotope-E2 proteins and DNA vectors of chimeric mimotope-E2 proteins.

Additionally, antigen combinations were found that were more effective in eliciting cross-reacting antibodies when present on a single conjugate than when present in a cocktail containing individual conjugates. Such antigen combinations are preferably produced using simultaneous conjugation techniques.

HCV conjugates are useful for eliciting an immune response in a subject. The immune response results in the production of antibodies recognizing HCV. The production of the immune response and the resulting antibodies can be employed in therapeutic or diagnostic applications.

A “subject” refers to a mammal capable of producing antibodies. Examples of subjects include humans, chimpanzees, mice and horses. Preferred subjects are humans. Other subjects, even those that do not become infected with HCV, can be used to generate anti-HCV antibodies.

Diagnostic applications include using HCV antibodies for detecting the presence of HCV in a test subject. Such antibodies can be obtained from subjects that have been induced to generate an immune response using an HCV conjugate described herein.

Therapeutic applications include treating a subject infected with HCV and prophylactically treating a subject. Such applications involve the use or production of HCV neutralizing antibodies. The ability of HCV conjugates described herein to produce antibodies reacting with different strains and variants of HCV is useful for treating or hindering initial infections of HCV, and for guarding against mutated forms of HCV produced in vivo.

Immunogenic HCV Peptide

An immunogenic HCV peptide comprises one or more HCV mimotopes and may also contain additional groups. Preferred additional groups are those enhancing an immune response and those involved in conjugation to a carrier. Additional groups that may be useful in enhancing an immune response include immunogenic peptides such as those containing an immunostimulatory invasin domain or a helper T cell epitope. (E.g., see, Wang, International Publication Number WO 99/66957 and Wang, U.S. Pat. No. 5,759,551.)

Methods of selecting for HCV mimotopes and particular mimotopes are described by Nicosia, et al., WO 99/60132 and Puntoriero, et al., EMBO Journal 17:3521-3533, 1998 (both of which are hereby incorporated by reference herein). Once identified, HCV mimotopes can be produced and incorporated into an HCV conjugate using standard conjugation techniques.

Immunogenic HCV peptides can be made up of common amino acids found in proteins and unnatural amino acids not normally found in proteins. Common amino acids found in proteins are the Lenantiomer (except for glycine which does not have a chiral center). Standard nomenclature used to reference common amino acids found in proteins is as follows:

A=Ala=Alanine;

C=Cys=Cysteine;

D=Asp=Aspartic acid;

E=Glu=Glutamic acid;

F=Phe=Phenylalanine;

G=Gly=Glycine;

H=His=Histidine;

I=Ile=Isoleucine;

K=Lys=Lysine;

L=Leu=Leucine;

M=Met=Methionine;

N=Asn=Asparagine;

P=Pro=Proline;

Q=Gln=Glutamine;

R=Arg=Arginine;

S=Ser=Serine;

T=Thr=Threonine;

V=Val=Valine;

W=Trp=Tryptophan; and

Y=Tyr=Tyrosine.

HCV conjugates may contain one or more different immunogenic HCV peptides. Preferably, at least one immunogenic HCV peptide comprises, consists essentially of, or consists of, an HCV mimotope sequence that is either SEQ. ID. NO. 1, SEQ. ID. NO. 2, SEQ. ID. NO. 3, SEQ. ID. NO. 4, SEQ. ID. NO. 5, SEQ. ID. NO. 6 or SEQ. ID. NO. 7. Reference to “consists essentially” indicates that additional groups may be present that do not substantially effect the ability of the mimotope to elicit an immune response. Examples of additional groups encompassed by reference to consists essentially of include reporter amino acids and groups involved in conjugation.

Sequences and designations used in the Examples provided below for SEQ. ID. NOs. 1-7 are as follows:

| | | |
|---|---|---|
| F78- | XTHTTGGQAGHQAHSLTGLFSPGAKQN, | SEQ. ID. NO. 1 |
| D6- | XTTTTGGQVSHATHGLTGLFSLGPQQK, | SEQ. ID. NO. 2 |
| R9- | XTTVVGGSQSHTVRGLTSLFSPGASQN, | SEQ. ID. NO. 3 |
| H1- | XTHTTGGVVGHATSGLTSLFSPGPSQK, | SEQ. ID. NO. 4 |
| R6- | TTTTTGGQVGHQTSGLTGLFSPGAQQN, | SEQ. ID. NO. 5 |
| N5- | TTTTTGGVQGHTTRGLVRLFSLGSKQN, | SEQ. ID. NO. 6 |
| and | | |
| M63- | XTHTTGGVVSHQTRSLVGLFSPGPQQN. | SEQ. ID. NO. 7 |

Each X for SEQ. ID. NOs. 1-7 is either glutamine or pyroglutamate, preferably glutamine. Pyroglutamate was found to occasionally form spontaneously from N-terminal glutamine in a peptide of SEQ. ID. NOs. 3.

In an embodiment of the present invention immunogenic HCV peptides comprise, consist essentially of, or consist of, the following sequences:

| | |
|---|---|
| XTHTTGGQAGHQAHSLTGLFSPGAKQNX¹X²; | SEQ. ID. NO. 8 |
| XTTTTGGQVSHATHGLTGLFSLGPQQKX¹X²; | SEQ. ID. NO. 9 |
| XTTVVGGSQSHTVRGLTSLFSPGASQNX¹X²; | SEQ. ID. NO. 10 |
| XTHTTGGVVGHATSGLTSLFSPGPSQKX¹X²; | SEQ. ID. NO. 11 |
| TTTTTGGQVGHQTSGLTGLFSPGAQQNX¹X²; | SEQ. ID. NO. 12 |
| TTTTTGGVQGHTTRGLVRLFSLGSKQNX¹X²; | SEQ. ID. NO. 13 |
| and | |
| XTHTTGGVVSHQTRSLVGLFSPGPQQNX¹X²; | SEQ. ID. NO. 14 |

SEQ. ID. NOs. 8-14 corresponds to SEQ. ID. NOs. 1-7 with the addition of X¹X², where X¹ is a reporter amino acid and X² is a sulfur containing amino acid. Each X for SEQ. ID. NOs. 8-14 is either glutamine or pyroglutamate, preferably glutamine.

A reporter amino acid is an amino acid other than one of the twenty common amino acids listed above. Examples of reporter amino acids are as follows: Nva—norvaline; Nle—norleucine; Abu—2-aminobutyric acid; Dbu—2,4-diaminobutyric acid; Dpr—2,3 diaminopropionic acid; and Chg—cyclohexylglycine.

A sulfur containing amino acid contains a reactive sulfur group. Examples of sulfur containing amino acids include cysteine and unnatural amino acids such as homocysteine. Additionally, the reactive sulfur may exist in a disulfide form prior to activation and reaction with carrier.

In another embodiment of the present invention immunogenic HCV peptides comprise, consist essentially of, or consist of, the following sequences:

| | |
|---|---|
| XTHTTGGQAGHQAHSLTGLFSPGAKQNX³C; | SEQ. ID. NO. 15 |
| XTTTTGGQVSHATHGLTGLFSLGPQQKX³C; | SEQ. ID. NO. 16 |
| XTTVVGGSQSHTVRGLTSLFSPGASQNX³C; | SEQ. ID. NO. 17 |
| XTHTTGGVVGHATSGLTSLFSPGPSQKX³C; | SEQ. ID. NO. 18 |
| TTTTTGGQVGHQTSGLTGLFSPGAQQNX³C; | SEQ. ID. NO. 19 |
| TTTTTGGVQGHTTRGLVRLFSLGSKQNX³C; | SEQ. ID. NO. 20 |
| and | |
| XTHTTGGVVSHQTRSLVGLFSPGPQQNX³C. | SEQ. ID. NO. 21 |

SEQ. ID. NOs. 15-21 correspond to SEQ. ID. NOs. 8-14 were the X¹ reporter group (shown in SEQ. ID. NOs. 15-21 as X³) is either norvaline, norleucine, 2-aminobutyric acid, 2,4-diaminobutyric acid, 2,3 diaminopropionic acid, or cyclohexylglycine; and the X² sulfur containing amino acid is cysteine. Each X for SEQ. ID. NOs. 15-21 is either glutamine or pyroglutamate, preferably glutamine.

Examples of an immunogenic HCV peptide containing more than one mimotope is provided by SEQ. ID. NOs. 22 and 23. SEQ. ID. NOs. 22 and 23, which contains mimotopes of both SEQ. ID. NOs. 1 and 7 are as follows:

| | |
|---|---|
| XTHTTGGQAGHQAHSLTGLFSPGAKQN | SEQ. ID. NO. 22 |
| QTHTTGGVVSHQTRSLVGLFSPGPQQNX¹X²; | |
| XTHTTGGQAGHQAHSLTGLFSPGAKQN | SEQ. ID. NO. 23 |
| QTHTTGGVVSHQTRSLVGLFSPGPQQNX³C; | |

where X, X¹, X² and X³ is as described above.

Peptides can be produced using techniques well known in the art. Such techniques include chemical and biochemical synthesis. Examples of techniques for chemical synthesis of polypeptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Polypeptide or Protein Complex Carrier

HCV mimotopes are covalently joined to a polypeptide or protein complex carrier. Carriers can be used to provide a scaffold for producing conjugates containing multiple antigens and to enhance the immune response.

Preferred polypeptide and protein complex carriers are immunogenic carriers able to enhance an immune response. Enhancement of the immune response can come from, for example, providing T helper cell epitopes or providing adjuvant activity. Examples of immunogenic carriers include the Outer Membrane Protein Complex of Neisseria meningitidis (OMPC), human serum albumin, tetanus toxoid, MMP-derived from OMPC, diptheria toxoid, hepatitis B virus surface antigen, hepatitis B virus core antigen, and human rotavirus VP6 capsid protein.

A preferred carrier is OMPC. OMPC contains numerous groups available for conjugation. The availability of a group for conjugation includes the type of group present and the position of the group in OMPC. Nucleophilic functionalities available for conjugation can be determined using techniques well know in the art. (See Emini, et al. U.S. Pat. No. 5,606,030.) One type of group that can be used for conjugation is primary amino groups present on amino acids such as the epsilon amino group of lysine and the alpha amino group of protein N-terminal amino acids. OMPC can be obtained using techniques well known in the art such as those described by Fu, U.S. Pat. No. 5,494,808.

Immunogenic HCV Peptide Combinations

The present application identifies preferred combinations of HCV mimotopes, and a preferred mimotope, that can be used to elicit antibodies cross reacting to different strains and variants of HCV. While combinations of mimotopes can be used as cocktails of different conjugates, it appears that greater efficacy can be achieved by producing a multiple conjugate containing a combination of different mimotopes.

Multiple Conjugates

In a preferred embodiment, the conjugate comprises, consists essentially or, consists of, two or more different types of immunogenic HCV peptides joined to the carrier, one or more capping groups joined to the carrier, and may contain one or more anions joined to the carrier. In additional embodiments, at least three different types of immunogenic HCV peptides are present, three different types of immunogenic HCV are present, four different types of immunogenic HCV peptides are present, five different types of immunogenic HCV peptides are present, or six different types of immunogenic HCV peptides are present.

Multiple conjugates preferably contain two or more immunogenic HCV peptides that comprise or consist essentially of a different HCV mimotope sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7. Each of the immunogenic HCV peptides can be joined to the carrier through an independently selected covalent linker. Preferably, such peptides are conjugated to OMPC as described in the structure below.

Examples of immunogenic HCV peptide combinations include those comprising the following sequence: (1) SEQ. ID. NOs. 1, 2, and 4; (2) SEQ. ID. NOs. 1, 3, and 5; (3) SEQ. ID. NOs. 1, 2, 3,4, and 5; (4) SEQ. ID. NOs. 1, 3,4, 5, and 6; (5) SEQ. ID. NOs. 1, 3, 4, 5, and 7; (6) SEQ. ID. NOs. 2, 3, 4, 5, and 22; and (7) SEQ. ID. NOs. 1, 2, 3, 4, 5 and 7. Preferably, such peptide combinations are conjugated to OMPC as described in the structure below.

In an embodiment of the present invention describing conjugation to OMPC, the conjugate has the following structure:

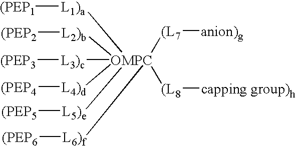

wherein OMPC represents the Outer Membrane Protein Complex of Neisseria meningitidis;

anion is a low molecular weight moiety having an anionic character at physiological pH; preferably, the anion is an ionized form of carboxylic, sulfonic, propionic or phosphonic acid;

L₁ is a covalent linker joining PEP₁ to OMPC, wherein each L₁ that is present may be the same or different;

L₂ is a covalent linker joining PEP₂ to OMPC, wherein each L₂ that is present may be the same or different;

L₃ is a covalent linker joining PEP₃ to OMPC, wherein each L₃ that is present may be the same or different;

L₄ is a covalent linker joining PEP₄ to OMPC, wherein each L₄ that is present may be the same or different;

L₅ is a covalent linker joining PEP₅ to OMPC, wherein each L₅ that is present may be the same or different;

L₆ is a covalent linker joining PEP₆ to OMPC, wherein each L₆ that is present may be the same or different;

L₇ is a covalent linker joining anion to OMPC, wherein each L₇ and each anion that is present may be the same or different;

L₈ is a covalent linker joining capping group to OMPC, wherein each L₈ and each capping group that is present may be the same or different;

PEP₁, PEP₂, PEP₃, PEP₄, PEP₅, and PEP₆, are immunogenic peptides, provided that at least two of PEP₁, PEP₂, PEP₃, PEP₄, PEP₅, and PEP₆ comprise a sequence selected from the group consisting of SEQ. ID. NO. 1, SEQ. ID. NO. 2, SEQ. ID. NO. 3, SEQ. ]D. NO. 4, SEQ. ID. NO. 5, and SEQ. ID. NO. 6;

a, b, c, d, e, f, g, and h is each an individually selected coupling load, wherein a, b, c, and h are each greater than 0;

or a pharmaceutically acceptable salt thereof.

A capping group is a chemical moiety that can react with a linker reactive group and inhibits the ability of the group to which it is attached to undergo further reaction. Suitable capping groups can readily be obtained for different linkers. For example, suitable capping groups for maleimide activated OMPC include thiol containing compounds such as N-acetylhomocysteine, n-acetylcysteine, and mercaptoethanol. Different types of capping groups can be employed including those that are, or not, anionic in character. Preferably, the capping groups present in a conjugate have the same chemical structure.

The “coupling load” is equal to the moles of a particular group divided by moles of lysine times 100. Preferably, the coupling load of a+b+c+d+e+f+g+h is about 25. Overall, the coupling load of a+b+c+d+e+f is preferably about 5 to about 20, wherein the coupling load of individual members that are present is preferably at least about 0.2. In different embodiments the overall coupling load a +b+c+d+e+f is at least 6, 7, or 8 and is no greater than 18, 19, or 20; and the coupling load of a, b, c, d, e, and f, when present (i.e., greater than zero), is individually at least about 0.4, preferably at least about 1.

Preferably, each of L₁, L₂, L₃, L₄, L₅, L₆, L₇, and L₈ that are present independently have the structure:

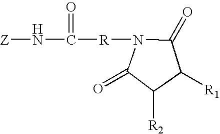

wherein OMPC is joined at position “z”,

R is selected from the group consisting of alkylene, substituted alkylene, and phenyl;

one of R₁ and R₂ is either hydrogen, alkyl, substituted alkyl or —SO₃H, and the other of R₁ and R₂ is the position to which PEP₁, PEP₂, PEP₃, PEP₄, PEP₅, PEP₆, anion or capping group is joined; preferably one of R₁ and R₂ is hydrogen; and

each anion is either carboxylic, sulfonic, propionic, or phosphonic acid.

“Alkylene” refers to a hydrocarbon group containing only carbon-carbon single bonds between the carbon atoms. The alkylene hydrocarbon group contains 1 to 12 carbon atoms and may be straight-chain or contain one or more branches or cyclic groups. The alkylene group is attached at two locations to other functional groups or structural moieties. Preferably, the alkylene group is either —(CH₂)₀₋₃—C₆H₁₀—(CH₂)₀₋₃—, or —(CH₂)₀₋₃—C₅H₈—(CH₂)0-3—, more preferably, the alkylene is either —C₆H₁₀—, or —C₆H₁₀—CH₂—.

“Substituted alkylene” refers to an alkylene where one or more of the hydrogens is replaced with a moiety that is either —NH₂, —NHCOCH₃, alkyl amino, carboxy, carboxy alkyl, sulphono, phosphono, halogen, —OH, —CN, —SH, —NO₂, —C₁₋₂ alkyl substituted with 1 to 5 halogens, —CF₃, —OCH₃, or —OCF₃.

“Alkyl” refers to a hydrocarbon group consisting of 1 to 8 carbon atoms joined by carbon-carbon single bonds. The alkyl hydrocarbon group may be straight-chain or contain one or more branches or cyclic groups. Preferably, the alkyl is one to four carbon atoms.

“Substituted alkyl” refers to an alkyl where one or more of the hydrogens is replaced with a moiety that is either —NH₂, —NHCOCH₃, alkyl amino, carboxy, carboxy alkyl, sulphono, phosphono, halogen, —OH, —CN, —SH, —NO₂, —C₁₋₂ alkyl substituted with 1 to 5 halogens, —CF₃, —OCH₃, or —OCF₃.

Preferably, each of PEP₁, PEP₂, PEP₃, PEP₄, PEP₅, and PEP₆ consists of a different sequence selected from the group consisting of SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14 and SEQ. ID. NO. 22, or from the group consisting of SEQ. ID. NO. 15, SEQ. ID. NO. 16, SEQ. ID. NO. 17, SEQ. ID. NO. 18, SEQ. ID. NO. 19, SEQ. ID. NO. 20, SEQ. ID. NO. 21 and SEQ. ID. NO. 23; and is joined to OMPC though the sulfur on its carboxy terminus amino acid.

In a more preferred embodiment each of L₁, L₂, L₃, L₄, L₅, L₆, L₇, and L₈ that are present independently have the following structure:

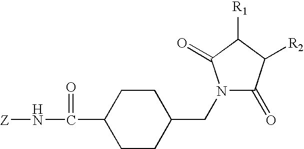

wherein OMPC is joined at position “z”, and R₁ and R₂ are as described above. Preferably, g is zero (i.e., the anion and L₇ are not present).

M63 Conjugate

In another preferred embodiment the HCV conjugate contains a polypeptide or protein complex carrier and comprises, consists essentially of, or consists of, (1) an immunogenic HCV peptide that comprises, consists essentially of, or consists of, the HCV mimotope sequence of SEQ. ID. NO. 7; (2) one or more capping groups joined to the carrier: and (3) may contain one or more anions joined to the carrier.

In an embodiment of the present invention the conjugate has the following structure:

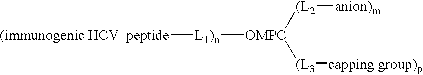

wherein OMPC represents the Outer Membrane Protein Complex of Neisseria meningitidis;

each anion is a low molecular weight moiety having an anionic character at physiological pH; preferably, the anion is an ionized form of carboxylic, sulfonic, propionic or phosphonic acid;

L₁ is a covalent linker joining the immunogenic HCV peptide to OMPC, wherein each L₁ may be the same or different;

L₂ is a covalent linker joining the anion to OMPC, wherein each L₂ and each anion that is present may be the same or different;

L₃ is a covalent linker joining the capping group to OMPC, wherein each L₃ and each capping group that is present may be the same or different;

n, m, and p is each an individually selected coupling load, wherein n and p are each greater than 0;

or a pharmaceutically acceptable salt thereof.

Preferably, the coupling load of n+m+p is about 25. In different embodiments n is at least about 0.4, at least about 1, at least about 3, at least 5, or at least about 8 and is no greater than about 20 or about 18.

In a preferred embodiment each of L₁, L₂ and L₃ independently contains the following structure:

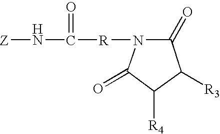

wherein OMPC is joined at position “z”,

R is selected from the group consisting of alkylene, substituted alkylene, and phenyl; and

one of R₃ and R₄ is either hydrogen, alkyl, substituted alkyl or —SO₃H, and the other of R₃ and R₄ is the position to which the immunogenic peptide, anion, or capping group binds; preferably one of R₃ and R₄ is hydrogen.

Alkylene, substituted alkylene, alkyl, and substituted alkyl are as described above, including preferred groups. Preferably, the immunogenic HCV peptide consists of SEQ. ID. NO. 14 or SEQ. ID. NO. 20 and is joined to OMPC though the sulfur group of its carboxy terminus amino acid.

In a more preferred embodiment L₁, L₂ and L₃ independently have the following structure:

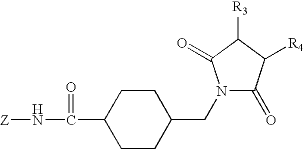

wherein R₃, R₄, and “z” are as described above.

In different embodiments of the present invention each anion that is present is either carboxylic, sulfonic, or phosphoric acid, and no anions are present (m is zero and L₂ is not present).

Polypeptide or Protein Complex Carrier Formation

Immunogenic HCV peptides containing HCV mimotopes can be conjugated to polypeptide and protein complex carriers through a covalent linker using conjugation techniques well known in the art. (See, Tolman, et al., U.S. Pat. No. 5,274,122, Emini, et al., U.S. Pat. No. 5,606,030, and Conley, et al., U.S. Pat. No. 5,763,574.) The conjugates can also be modified, for example, to contain one or more anionic groups.

A covalent linker joining an immunogenic HCV peptide or anion to a carrier is stable under physiological conditions. Examples of such linkers are non-specific cross-linking agents, monogeneric spacers and bigeneric spacers.

Non-specific cross-linking agents and their use are well known in the art. Examples of such reagents and their use include reaction with glutaraldehyde; reaction with N-ethyl-N′-(3-dimethylaminopropyl) carbodiimide, with or without admixture of a succinylated carrier; periodate oxidation of glycosylated substituents followed by coupling to free amino groups of a protein carrier in the presence of sodium borohydride or sodium cyanoborohydride; diazotization of aromatic amino groups followed by coupling on tyrosine side chain residues of the protein; reaction with isocyanates; or reaction of mixed anhydrides. See, generally, Briand, et al., J. Imm. Meth. 78:59, 1985.

Monogeneric spacers and their use are well known in the art. Monogeneric spacers are bifunctional and require functionalization of only one of the partners of the reaction pair before conjugation takes place. An example of a monogeneric spacer and its use involves coupling an immunogenic HCV peptide to one end of the bifunctional molecule adipic acid dihydrazide in the presence of carbodiimide. A diacylated hydrazine presumably forms with pendant glutamic or aspartic carboxyl groups of the carrier. Conjugation then is performed by a second coupling reaction with carrier protein in the presence of carbodiimide.

Bigeneric spacers and their use are well known in the art. Bigeneric spacers are formed after each partner of the reaction pair is functionalized. Conjugation occurs when each functionalized partner is reacted with its opposite partner to form a stable covalent bond or bonds. (See, for example, Marburg, et al., J. Am. Chem. Soc. 108:5282-5287, 1986; and Marburg, et al., U.S. Pat. No. 4,695,624.)

Examples of different processes include the following:

### Process 1

1a. React a fraction of the protein carrier nucleophilic groups with a reagent to generate a thiol group. An example of such a reagent is N-acetyl homocysteine thiolactone;

1b. React the product of step 1a with a reagent comprising an electrophile and an anion having a negative charge at physiological pH. An example of such a reagent is maleimidoalkanoic acid; and

1c. React the product of step 1b with immunogenic peptides previously derivatized so as to append an electrophilic group.

### Process 2

2a. React a fraction of the protein carrier nucleophilic groups with a bifunctional electrophilic reagent to generate an electrophilic protein. An example of such a reagent is maleimidoalkanoic acid hydroxysuccinimide ester;

2b. React the product from step 2a with a reagent comprising both a nucleophile and an anion. An example of such a reagent is α-mercaptoacetic acid; and

2c. React the product of step 2b with immunogenic peptides containing a nucleophile, such as a thiol group.

### Process 3

3a. React a fraction of the protein nucleophilic carrier groups with a reagent comprising both an electrophile and an anion or incipient anion. Examples of such reagents include N-(bromoacetyl)-6-amino caprole acid, and succinic anhydride;

3b. React the residual fraction of nucleophilic groups on the product of step 3a with a reagent, for example with N-acetyl homocysteine thiolactone, which generates thiol groups on the protein carrier; and

3c. React the product of step 3b with immunogenic peptides previously derivatized so as to append an electrophilic group, for example, a group comprising maleimide.

### Process 4

4a. React a fraction of the protein carrier nucleophilic groups with a reagent comprising both an electrophile and an anion or incipient anion, such as N-(bromoacetyl)-6-amino caproic acid or succinic anhydride;

4b. React the residual protein nucleophilic groups on the product of step 4a with a bifunctional electrophilic reagent to append electrophilic sites onto the protein. Examples of such reagents are maleimidoalkanoic acid hydroxysuccinimide ester; and

4c. React the product of step 4b with immunogenic peptides containing a nucleophilic group, such as a thiol.

### Process 5

5a. React a fraction of the protein carrier amino groups with a crosslinker having the structure:

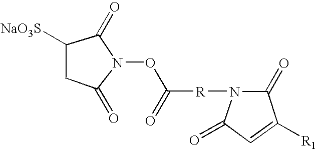

wherein R is selected from the group consisting of alkylene, substituted alkylene, and phenyl; and R₁ is selected from the group consisting of hydrogen, alkyl, substituted alkyl, and —SO₃H; where alkylene, substituted alkylene, alkyl, and substituted alkyl are as described above, including preferred groups;

5b. React the product of step 5a with immunogenic peptides containing a nucleophilic group, such as a thiol.

Processes 1-5 will generally involve additional steps such as removal of excess reagent from one step prior to commencing a second step and the capping of unreacted functionalities. The Reaction Scheme provided below illustrates the use of process 5 along with additional steps to produce a conjugate. The components listed in the Reaction Scheme such as the carrier, crosslinker, capping groups, and mimotope are for illustration purposes. Based on the present disclosure, one of ordinary skill in the art can replace the components in the Reaction Scheme with components well known in the art and components described herein.

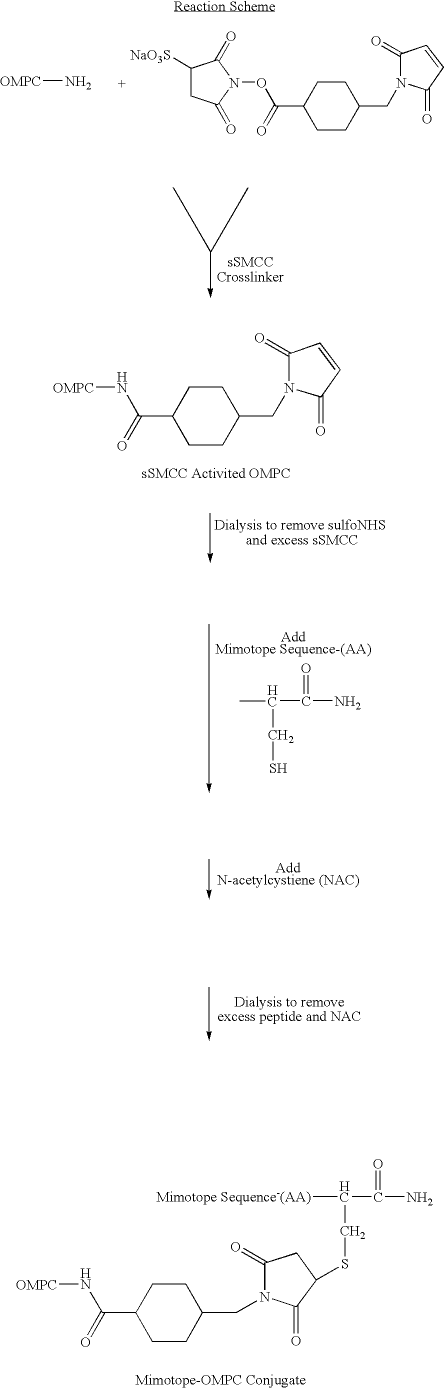

Formulation and Administration

HCV conjugates can be formulated and administered to a subject using the guidance provided herein along with techniques well known in the art. Guidelines for pharmaceutical administration in general are provided in, for example, Modern Vaccinology, Ed. Kurstak, Plenum Med. Co. 1994; Remington's Pharmaceutical Sciences 18^{th} Edition, Ed. Gennaro, Mack Publishing, 1990; and Modern Pharmaceutics 2^{nd} Edition, Eds. Banker and Rhodes, Marcel Dekker, Inc., 1990, each of which are hereby incorporated by reference herein.

HCV conjugates can be prepared as acidic or basic salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine.

Doses of HCV conjugates can be administered by different routes such intravenous, intraperitoneal, subcutaneous, or intramuscular. A preferred route is intramuscular.

Suitable dosing regimens are preferably determined taking into account factors well known in the art including age, weight, sex and medical condition of the subject; the route of administration; the desired effect; and the particular compound employed. The conjugates can be used in multi-dose vaccine formats. It is expected that a dose would consist of the range of 1 μg to 1.0 mg total protein, in an embodiment of the present invention the range is 0.1 mg to 1.0 mg.

The HCV conjugate is preferably formulated with an adjuvant. Examples of adjuvants are alum, AlPO₄, alhydrogel, Lipid-A and derivatives or variants thereof, Freund's complete or incomplete adjuvant, neutral liposomes, liposomes containing vaccine and cytokines or chemokines.

The timing of doses depend upon factors well known in the art. After the initial administration one or more booster doses may subsequently be administered to maintain antibody titers. An example of a dosing regime would be day 1, 1 month, a third dose at either 4, 6 or 12 months, and additional booster doses at distant times as needed.

### EXAMPLES

Examples are provided below to further illustrate different features of the present invention. The examples also illustrate useful methodology for practicing the invention. These examples do not limit the claimed invention.

### Example 1

Peptide Synthesis

Peptides were custom synthesized as linear sequences by solid phase FMOC chemistry methods (using commercial sources). All peptides were >90% purity as judged from RP-HPLC. In general, each peptide consisted of 29 residues, including a 27-mer mimotope, a carboxy penultimate unnatural residue, and a carboxy terminal cysteine present as a carboxamide. Examples of such peptides include the following:

| | |
|---|---|
| XTHTTGGQAGHQAHSLTGLFSPGAKQNX³C | SEQ. ID. NO. 24 |
| (where X³ is Nle), | |
| XTHTTGGQAGHQAHSLTGLFSPGAKQNX³C | SEQ. ID. NO. 25 |
| (where X³ is Chg), | |
| XTTTTGGQVSHATHGLTGLFSLGPQQKX³C | SEQ. ID. NO. 26 |
| (where X³ is Nva), | |
| XTTVVGGSQSHTVRGLTSLFSPGASQNX³C | SEQ. ID. NO. 27 |
| (where X³ is Dpr), | |
| XTHTTGGVVGHATSGLTSLFSPGPSQKX³C | SEQ. ID. NO. 28 |
| (where X³ is Abu), | |
| TTTTTGGQVGHQTSGLTGLFSPGAQQNX³C | SEQ. ID. NO. 29 |
| (where X³ is Dbu), | |
| TTTTTGGVQGHTTRGLVRLFSLGSKQNX³C | SEQ. ID. NO. 30 |
| (where X³ is Nle), | |
| XTHTTGGVVSHQTRSLVGLFSPGPQQNX³C | SEQ. ID. NO. 31 |
| (where x³ is Nle). | |

Each X for SEQ. ID. NOs. 24-31 is either glutamine or pyroglutamate, preferably glutamine. The peptides were synthesized with X being glutamine.

For examining linkages at the amino terminus, peptides were synthesized as a 30-mer including an N-terminal glycine, cysteine, an unnatural residue, and a 27-mer mimotope sequence with the terminal residue present as a carboxamide.

For examining a cyclized mimotope peptide, the synthesis and cyclization followed a strategy published previously (Conley, et al., Vaccine 12:445-451, 1994). Briefly, the peptide was synthesized with an N-terminal glycine, cysteine, lysine, followed by the 27-mer mimotope sequence, an additional penultimate carboxy terminal aspartic acid, and the terminal unusual amino acid. Condensation between the ε-amino of lysine and the β-carboxyl of the aspartic acid yielded the cyclic product.

Custom synthesized hepatitis C virus region derived peptides (HVR) were used as ELISA antigens. These were prepared either without (Table 1A) or with (Table 1B) a biotin group for standard peptide adsorption or strepavidin biotin capture ELISA formats, respectively.

Table 1A. Non-Biotinylated HVR Peptides Used in Standard ELISA

!SEQ ID. NO.? Designation? Sequence

| | | |
|---|---|---|
| 32 | c882-01 | ETHVTGGNAGRTTAGLVGLLTPGAKQN |
| 33 | c882-02 | ETHVTGGSAGRTIAGFTGLLTPGAKQN |
| 34 | c882-03 | ETHVTGGSAGHTTAGLVGLLTPGAKQN |
| 35 | c882-04 | ETHVTGGSAGHTTAGLVRLLSPGAKQN |
| 36 | BK | NTRVTGGVQSHTTRGFVGMFSLGPSQR |
| 37 | 147-05 | ETHVTGGSAAYAAHGLANILSRGAKQD |
| 38 | 147-07 | TTYTTGGSAGHSLPGISNLFAPGAQQH |
| 39 | 147-08 | DTYTTGGSAARSTLGLTSLFVAGPKQN |
| 40 | 147-09 | FTRVTGGAQAVPTHGLTSLFTFGAQQN |
| 41 | 147-11 | NTYVTGGQAGYTTMALSSLFAPGAQQN |

Table 1B. Biotinylated HVR peptides used in the biotin-capture ELISA

| | | |
|---|---|---|
| SEQ ID. NO | Designation | Sequence |

| | | |
|---|---|---|
| 42 | H730 | ETHVTGGNAGRTTAGLVGLLTPGAKQN |
| 43 | H732 | ETHVTGGNAGRTTAGLVGLLTPDAKQN |
| 44 | h731 | ETHVTGGNAGRTAAGLVGLLTPGAKQN |
| 45 | 1012 | ETHVTGGSAGHTVSGFVSLLAPGAKQN |
| 46 | 1008 | NTYVTGGAAARGASGITSLFSRGPSQK |
| 47 | 1009 | GTTRVGGAAARTTSSFASLLTHGPSQN |
| 48 | 1010 | ETRVTGGAAGHTAFGFASFLAPGAKQN |
| 49 | 270 | DTHVVGGATERTAYSLTGLFTAGPKQN |
| 50 | 271 | GTTCQGGVYARGAGGIASLFSVGANQK |
| 51 | 272 | RTLSFGGLPGHTTHGFASLSAPGAKQN |
| 52 | 273 | RTILMAGRQAEVTQSFPGLFSLAPSQK |

### Example 2

Peptide Conjugates

N. meningitidis OMPC was activated at pH 8.0 (10 mM HEPPS buffer) by reaction with an excess of the heterobifunctional crosslinker sulfosuccinimidyl 4(N-maleimidomethyl) cyclohexane-1-carboxylate (sSMCC, Pierce Chemical Co., Rockford, Ill.) for 2 hours at 2 to 8° C. The activated OMPC was using 300K MWCO DispoDialyser, (Spectrum Laboratories, Inc., Rancho Dominguez, Calif.) against 10 mM HEPPS, pH 8.0 or 1.0 mM MOPS, pH 7.1 for 18 to 24 hours at 2 to 8° C., with three buffer changes, to remove sulfoNHS and excess sSMCC.

The individual reactions proceeded by the addition of each thiol-containing mimotope peptide. Unreacted maleimide groups on the OMPC then were reacted with N-acetylcysteine (NAC) followed by dialysis, 300K MVCO DispoDialyser, against 10 mM (N-2-hydroxyethyl piperazine-N′-3-propane sulfonic acid (HEPPS), pH 8.0, to remove excess unconjugated peptide and NAC.

The percent coupling load of mimotope peptide to total OMPC lysine was calculated from the ratio of the moles of unnatural amino acid in the mimotope over the moles of lysine multiplied by 100. Quantitation was based on amino acid analysis of the acid hydrolysed conjugate, 6 N HCl for 20 hours at 110° C. For lysine-containing mimotopes a correction was made to the observed lysine value to obtain the OMPC lysine contribution. Protein concentrations were determined by Lowry assay using BSA as a standard. SDS-PAGE analysis was performed to provide evidence of covalent linkage of the mimotope peptides to protein components of OMPC. Coupling loads were in the range of 8 to 19%.

Some of the vaccines studied were prepared with multiple mimotopes, either 3, 5, or 6 simultaneously conjugated. These were designated simultaneously conjugated peptide vaccines (SCPV). The mimotope components, and the “reporter” amino acid associated with each mimotope peptide, of these SCPV are given in Table 2. In order to determine the degree of peptide coupling to the OMPC in a SCPV preparation, additional unique “reporter” amino acids were incorporated as the C-terminal penultimate residue for each mimotope.

TABLE 2
Composition of Simultaneously Conjugated Peptide Vaccines

| | Number of | Designation- SEQ. ID. NO. (reporter amino |
|---|---|---|
| SCPV No. | Mimotopes | acid) Percent Coupled |
| 1 | 3 | D6- SEQ. ID. NO. 26 (Nva) |
| | | F78- SEQ. ID. NO. 24 (Nle) |
| | | H1- SEQ. ID. NO. 28 (Abu) |
| 1a | 3 | F78- SEQ. ID. NO. 24 (Nle) 3.9% |
| | | R6- SEQ. ID. NO. 29 (Dbu) 2.0% |
| | | R9- SEQ. ID. NO. 27 (Dpr) 2.6% |
| 2 | 5 | D6- SEQ. ID. NO. 26 (Nva) 3.9%, |
| | | F78- SEQ. ID. NO. 24 (Nle) 1.6%, |
| | | H1- SEQ. ID. NO. 28 (Abu) 1.5%, |
| | | R6- SEQ. ID. NO. 29 (Dbu) 2.7%, |
| | | R9- SEQ. ID. NO. 27 (Dpr) 7.2%. |
| 3 | 5 | F78- SEQ. ID. NO. 24 (Nle) |
| | | H1- SEQ. ID. NO. 28 (Abu) |
| | | N5- SEQ. ID. NO. 30 (Nle) |
| | | R6- SEQ. ID. NO. 29 (Dbu) |
| | | R9- SEQ. ID. NO. 27 (Dpr) |
| 3a | 5 | F78- SEQ. ID. NO. 25 (Chg) 0.8 to 1.0%, |
| | | R6- SEQ. ID. NO. 29 (Dbu) 2.0 to 3.1%, |
| | | R9- SEQ. ID. NO. 27 (Dpr) 2.9 to 4.9%, |
| | | H1- SEQ. ID. NO. 28 (Abu) 0.4 to 0.6%, |
| | | M63- SEQ. ID. NO. 31 (Nle) 1.7 to 2.5%. |
| 4 | 6 | D6- SEQ. ID. NO. 26 (Nva) 3.5%, |
| | | F78- SEQ. ID. NO. 24 (Nle) 2.3%, |
| | | M63- SEQ. ID. NO. 31 (Nle) 2.3%, |
| | | H1- SEQ. ID. NO. 28 (Abu) 1.2%, |
| | | R6- SEQ. ID. NO. 29 (Dbu) 2.2%, |
| | | R9- SEQ. ID. NO. 27 (Dpr) 7.3%. |

Abbreviations:
Nva - norvaline,
Nle - norleucine,
Abu - 2-aminobutyric acid,
Dbu - 2,4-diaminobutyric acid,
Dpr - 2,3 diaminopropionic acid,
Chg - cyclohexylglycine.

### Example 3

DNA Vector Immunogens

Six individual expression plasmids were constructed to elicit immune responses by DNA delivery to primates. The six mimotope sequences, SEQ. ID. NOs. 1, 3, 4, 5, 6, and 7 were individually appended to the ectodomain sequences of the HCV E2 of strain N (Hayashi, et al., J. Hepatol. 17:S94-S 107, 1993), to generate a chimeric protein. The HVR encoding sequences were removed from this E2 sequence and a (his)₆ tag was added to the carboxyl terminus. These constructs were cloned in the plasmid V1jnstpa in frame with the tissue plasminogen activator (tPA) leader sequences. (Montgomery, et al., Pharmacol. Ther. 74:195-205, 1997.)

All constructs were verified by DNA sequence analysis. Large scale plasmid preparations were prepared from transformed Escherichia coli strain DH5α cultures grown in Terrific broth, 1X salts, supplemented with 50 μg/ml kanamycin. After plasmid extraction by standard methods, DNA was banded twice in CsCl density gradients, dialyzed extensively against 10 mM Tris, pH 8.0, 1 mM EDTA and ethanol precipitated from solutions containing 200 mM Na acetate.

### Example 4

Glycoprotein Immunogens

Six chimeric mimotope-containing glycoproteins were produced as secreted products by transient expression in 293T cell cultures. Generally for expression, 293T cells were cultured in poly D-lysine-coated 175 cm² flasks (BioCoat, Becton-Dickinson) and were transfected at 60 to 70% confluence.

The DNA, at 1μg/82 l in sterile 10 mM Tris, 1 mM EDTA, pH 8.0, was mixed with the Lipofectamine Plus component of the Lipofectamine Plus kit (GIBCO), according to instructions, using approximately 25 μg DNA/flask. This solution was mixed with 2.0 ml of Opti-MEM containing 75 μl of lipofectamine and incubated according to instructions. This 4.0 ml was added to a flask already containing 15 ml of fresh Opti-MEM. After 5-6 hours the Opti-MEM based lipofectamine-DNA medium was removed and replaced with 24 ml of a protein-free medium supplemented with L-glutamine and penicillin/streptomycin (CELLGRO Free—complete serum, protein, hormone, growth factor free medium, CellGro, Herndon, Va.). This medium was harvested every 24 hours post-transfection for 3 harvests, clarified by low speed centrifugation, and stored at 4° C. until all materials were collected, pooled, and passed through a 0.22μ filter. The medium was then dialyzed against 10 mM imidazole, 0.5 M NaCl, 20 mM Na phosphate, pH 7.8 (starting buffer) and filtered again using a 0.22μ filter.

Approximately 2.0 L of material was loaded on a 10 ml volume Ni-NTA column (Qiagen, Valencia, Calif.) at a flow rate of approximately 3 ml/minute at 4° C. The column was washed with starting buffer, then eluted stepwise with: (a) 40 ml of buffer with 20 mM imidazole; (b) 10 ml of buffer with 40 mM imidazole; (c) 30 ml of buffer with 100 mM imidazole; and (d) 15 ml of buffer with 250 mM imidazole. Essentially all the chimeric glycoprotein eluded with the 100 mM imidazole buffer. Portions of each glycoprotein's major eluded fraction were dialyzed into water for N-terminal protein sequence analysis, phosphate buffered saline for ELISA studies, or 5 mM triethanolamine, 150 mM NaCl, pH 7.8 for subsequent vaccine formulation.

Purity was assessed by SDS-PAGE followed by western blot using either an anti-HCV gpE2 specific murine monoclonal antibody or an anti-tetra-his antibody (Qiagen, Valencia, Calif.). Identity and completion of leader processing was confirmed by N-terminal amino acid sequence analysis. Protein sequence analysis showed that cleavage of the tPA leader from all these proteins was between the leader residues 22 and 23, pro and ser, whereas the cleavage normally occurs between positions 23 and 24 (ser and gln) for native tPA (Siebert and Fong, Nucleic Acids Res. 18:1086, 1990).

### Example 5

Vaccine Formulation

The first studies in rabbits were carried out using peptide conjugate vaccines formulated in Freunds complete and incomplete adjuvants. Peptide OMPC conjugate vaccines were prepared in 500 μg total protein doses in 10 mM HEPPS, pH 8.0. Further studies then were performed with each dose adsorbed onto 1.4 mg/ml AlPO₄ in 6 mM triethanolamine saline pH 7.0-8.0 to which monophosphoryl lipid-A (MPL-A) was already adsorbed. The weight ratio of AlPO₄ to MPL-A was 3.5 to 1. The purified chimeric proteins were also prepared in 500 μg total protein doses in 5 mM triethanolamine, 150 mM NaCl, pH 7.8. Each dose was adsorbed onto alum with pre-adsorbed MPL-A at a weight ratio of alum to MPL-A of 3.5 to 1. DNA vector vaccines were resuspended in sterile PBS, pH 7.5 at 5.0 mg/nil.

### Example 6

Immunogenicity Studies

New Zealand white rabbits (2 to 2.5 Kg) were injected at the start of each study and at weeks 4 and 8. The animals were bled to prepare serum biweekly through week 12. Rhesus monkeys, Maccca mulatta (>2.0 Kg) were immunized with the mimotope OMPC conjugate vaccines and the chimeric mimotope glycoprotein vaccines at day 1 and weeks 4, 8, and 26.

Each dose was inoculated intramuscularly and equally distributed between 2 sites in each deltoid muscle. The monkeys were immunized with the DNA vector vaccines prepared in sterile PBS. Each animal was inoculated intramuscularly using a Biojector (Bioject, Inc. Portland, Oreg.) needle-less device fitted with a number 3 syringe. Each dose was equally distributed between two sites in each quadracep muscle. The animals were bled and sera were prepared biweekly through week 12, monthly through week 24, then biweekly again through week 32.

### Example 7

Western Blot Assay

Western blot (WB) assay of recombinant proteins was generally performed after sample preparation in 4× NuPage buffer and electrophoresis in preformed 4-12% NuPage gels (NOVEX, San Diego, Calif.). Separated proteins were transferred at 30 volts for 90 minutes to PVDF paper and processed by standard methods. Processed PVDF sheets were exposed to rabbit or monkey immune sera or monoclonal antibodies overnight at 4° C.

In general HRP-conjugated antisera were used as secondary antibodies. For detecting mouse monoclonal antibody, a goat anti-mouse IgG, Fc fragment specific antibody was used (Jackson Immuno. Res., West Grove, Pa.). For detecting rabbit antibodies, a goat anti rabbit IgG-Fc was used (Bethyl Labs, Montgomery, Tex.). For detecting monkey antibodies, a goat anti-monkey IgG H+L was used (Bethyl Labs). All these secondary antibodies were used at a 1:4000 dilution. All blots were developed on Hyperfilm using ECL or ECL+reagents (Amersham, Piscataway, N.J.).

### Example 8

ELISA of Immune Sera to Mimotopes and HVR Peptides

Antibody responses were determined against the homologous immunizing peptide, heterologous mimotopes, or HVR peptides by an enzyme-linked immunoadsorbent assay (ELISA) essentially as described (Tolman, et al., 1993. Int. J. Peptide Protein Res. 41:455-466, 1993 and Conley, et al., Vaccine 12:445-451, 1994) or by use of streptavidin capture of biotinylated peptide ELISA.

Briefly, for the standard ELISA, microtiter plates were coated with 0.25 μg of peptide in PBS in a humid atmosphere, overnight at 4° C. The plates were washed extensively, blocked with 1× blocker BSA (Reacti-Bind, Pierce, Rockford, Ill.), washed extensively, then reacted with dilutions of test and control sera for 1 hour at 37° C. The plates then were washed extensively and reacted to the conjugated secondary antibody. The plate was processed for reaction by standard methods, and absorbance was measured at 450 nm.

For the streptavidin capture of biotinylated peptide ELISA, 50 pmol of biotinylated peptide, 2× well capacity, was reacted to streptavidin coated ELISA plates (Reacti-Bind, Pierce, Rockford, Ill.) in blocker BSA at room temperature after sealing each plate. The plates then were washed extensively, blocked, washed again, and reacted with dilutions of test and control sera for 30 minutes at 37° C. The remaining methods of washing, reaction to secondary antibody, processing, and measurement again followed standard protocols.

### Example 9

Capture ELISA

We used an HCV E2 glycoprotein capture ELISA to measure anti-mimotope responses to recombinant glycoproteins. ELISA plates were coated with 50 pg of either of two murine anti-HCV E2 monoclonal antibodies overnight at 4° C. in a humid environment. These Mabs were prepared from hybridoma cell cultures using serum-free medium and purified using HiTrap Protein G (Amersham/Pharmacia, Piscataway, N.J.). These two antibodies appeared to bind to conserved determinants, located at a distance from the N-terminus of E2, and by analysis did not interfere with anti-mimotope or anti-HVR sera binding to E2 in ELISA or BIACore.

On day 2, the plates were washed, blocked, and the purified glycoproteins were captured. After washing again, the plates were reacted with dilutions of test and control sera for 1 hour at 37° C. The remaining methods of washing, reaction to secondary antibody, processing, and measurement again followed standard protocols.

### Example 10

Immunogenicity of Singular Mimotope Conjugates in Rabbits

Five individual vaccine HCV mimotope peptide conjugates were tested using four animals (Table 3). The endpoint titers, expressed as GMT, for responses to the homologous immunizing sequence ranged from 1:5.7×10⁵ for the R9 mimotope conjugate to 1:7.6×10⁶ for the R6 mimotope conjugate. Anti-peptide titers in this high range are consistent for rabbits using OMPC carriers and Freund's adjuvant. Four HCV H strain peptides (Farci, et al., 1996 P.N.A.S. 93:15394-15399) then were used to evaluate these anti-mimotope responses to HVR sequences.

TABLE 3
Immunogenicity of HCV E2 HVR Mimotope OMPC
Conjugates in Rabbits

| Mimotope- | PEPTIDE ELISA, RECIPROCAL OF GMT | | | | |
|---|---|---|---|---|---|
| OMPC | Cognate | HCV H | HCV H | HCV H | HCV H |
| vaccine | mimotope | HVR1 | HVR2 | HVR3 | HVR4 |
| D6-OMPC | 1.90 × 10⁶ | <640 | 20,000 | 5100 | 5,100 |
| R9-OMPC | 0.57 × 10⁶ | 82,000 | 82,000 | 84,000 | 82,000 |
| H1-OMPC | 1.40 × 10⁶ | <640 | <640 | <640 | <640 |
| R6-OMPC | 7.64 × 10⁶ | 82,000 | n.d. | 82,000 | 21,000 |
| F78-OMPC | 1.86 × 10⁶ | 82,000 | n.d. | 82,000 | 21,000 |

n.d. - not done.

Anti-mimotope R9 antibodies recognized 4 of 4 HVR peptides, while anti-mimotope R6 and F78 antibodies recognized 3 of 3 HVR peptides. On the other hand, anti-mimotope D6 antibodies only recognized the three variant sequences and were of low titer. The anti-mimotope H1 antibodies recognized none of the H strain variants.

Additional studies were performed in rabbits to determine the immunogenicity of conjugates when formulated using MPL-A. The conjugates and MPL-A were absorbed on AlPO₄ since both the complex and the formulation were stable. Within this framework three mimotope M63 conjugates were tested, a linear C-terminal conjugate, a linear N-terminal conjugate, and a cyclic peptide conjugate (Table 4).

TABLE 4
Immunogenicity Of Mimotope M63 Conjugate Vaccines In Rabbits

| M63 | Peptide ELISA, Reciprocal of GMT | | | | | | | |
|---|---|---|---|---|---|---|---|---|
| mimotope | M63 | M63 | F78 | HCV BK | HCV H | HCV H | HCV H | HCV H |
| vaccine | linear | cyclic | mimotope | HVR | HVR1 | HVR2 | HVR3 | HVR4 |
| C-terminal | 8.6 × 10⁷ | n.d. | 1.6 × 10⁶ | 1.55 × 10⁶ | 380 | <160 | 375 | 24,400 |
| conjugated | | | | | | | | |
| N-terminal | 6.5 × 10⁵ | n.d. | 6100 | 5920 | 5920 | <160 | 5920 | 95,000 |
| conjugated | | | | | | | | |
| Cyclic | 5.4 × 10⁵ | 1.6 × 10⁶ | 10,240 | 140 | 5910 | <160 | 5920 | 5920 |
| peptide | | | | | | | | |
| conjugated | | | | | | | | |

The responses, as endpoint titer, to the cognate M63 peptide were similar for the linear C-terminal version when compared to the previous conjugates that were formulated in Freund's adjuvant. Additionally we found that endpoint titers to the heterologous mimotope P78 and the HCV BK strain HVR peptide were >1:1.5×10⁶. Thus, the M63 mimotope appears to have the capability, under some conditions, to elicit antibodies that are high titer in their reactivity to heterologous sequences. The immune sera from animals immunized with the N-terminal or the cyclic peptide conjugates had considerably lower endpoint titer to the M63 mimotope peptide, approximately 100-fold lower, yet these sera recognized more of the HCV H strain HVR sequences than the sera elicited by the C-terminal conjugate.

### Example 11

Immunogenicity of SCPV in Rabbits

A mixture of five individual conjugates and four different multiple mimotope vaccines were evaluated in rabbit immunogenicity studies. The mixture of five individual conjugate vaccines elicited ELISA responses to the five cognate peptides in the range of 1:6.5×10⁵ to 1:2.6×10⁶ (Table 5).

TABLE 5
Immunogenicity of Multiple Mimotope Vaccine in Rabbits

| | Peptide ELISA, Reciprocal of GMT | | | | | | |
|---|---|---|---|---|---|---|---|
| Mimotopes | Preparation | Cognate | HCV BK | HCV H | HCV H | HCV H | HCV H |
| in vaccine | method | mimotopes | HVR | HVR1 | HVR2 | HVR3 | HVR4 |
| D6, R9, | Mixed | 0.65 to 2.6 × 10⁶ | n.d. | 20,487 | <160 | 23,648 | <160 |
| H1, R6, | | | | | | | |
| F78 | | | | | | | |
| D6, H1, | SCPV | 0.16 to 0.65 × 10⁶ | 10,240* | 21,187 | 7,460 | 29,070 | 21,189 |
| F78 | | | | | | | |
| D6, R9, | SCPV | 0.041 to 2.6 × 10⁶ | 338,432 | 21,115 | 5,296 | 82,090 | 1,865 |
| H1, R6, | | | | | | | |
| F78 | | | | | | | |
| D6, R9, | SCPV | 0.0025 to 2.6 × 10⁶ | 117,760 | 20,522 | 1,283 | 81,930 | 1,283 |
| H1, R6, | | | | | | | |
| F78, M63 | | | | | | | |

* Tests were performed with purified gamma globulin.

For the different mixtures, the ELISA titers for the HCV H strain HVR variant peptides showed a response to variant #1 and #3 only from one of the rabbits. The remainder of the sera did not recognize any of the HCV H strain HVR sequences.

Three different types of SCPV were prepared, tested, and the results compared to the mixed mimotope immunized animals. The three SCPVs consisted of one vaccine containing three mimotopes, one containing five mimotopes and a third containing six mimotopes.

The peptide ELISA studies again showed that SCPVs could elicit high titer anti-mimotope responses (Table 5). The ranges of GMT to the mimotopes in the single mimotope vaccine studies and in the multiple mimotope vaccine studies overlapped from a low of 1:2500 against R9 mimotope, in the six SCPV study, to a high of near 1:7.5×10⁶ in the R6 single mimotope study. The R9 peptide was the highest coupled peptide in both SCPV number 2 and number 4 (Table 2).

For all forms of multiple mimotope vaccines, the GMT observed for reactivity to the major HCV H strain HVR were near 1:20,000. In contrast to the mixed vaccines, the SCPV sera reacted consistently to the variant sequences and with high titer to the BK isolate HVR sequence. Considering all the ELISA titers it appeared that a SCPV using five mimotope peptides elicited the broadest reactivity patterns.

### Example 12

Immunogenicity of OMPC Conjugates, Chimeric Glycoproteins, and DNA Vector of Those Chimeric Glycoproteins in Rhesus Monkeys

Rhesus monkeys were used in primate immunogenicity studies and to compare three ways to deliver mimotope based vaccines with the goal of eliciting broadly HVR-reactive antibodies. Peptide conjugates were prepared for five singular mimotopes, one SCPV consisting of five mimotopes, and a conjugate consisting of the HVR sequence of HCV strain BK. N5 conjugation occurred at a low efficiency.

The results of a peptide ELISA, ELISA by capture of biotinylated peptide, and ELISA by Mab capture of recombinant chimeric E2 are shown in Tables 6A-6C.

TABLE 6A
Immunogenicity of Mimotope OMPC Conjugate Vaccine: Peptide ELISA,
Reciprocal of GMT

| Mimotope- | | HCV | | | | | | | | | |
|---|---|---|---|---|---|---|---|---|---|---|---|
| OMPC | Cognate | BK | HCV H | HCV H | HCV H | HCV H | 147- | 147- | 147- | 147- | |
| vaccine | mimotope | HVR1 | HVR1 | HVR2 | HVR3 | HVR4 | 05 | 07 | 08 | 09 | 147-11 |
| F78 | 6270 | <40 | <40 | <40 | <40 | 2,090 | <40 | <40 | <40 | <40 | <40 |
| M63 | 57,900 | 1525 | 380 | 130 | 390 | 130 | <40 | <40 | <40 | <40 | <40 |
| R6 | 40,960 | <40 | <40 | <40 | <40 | <40 | <40 | 2090 | 1480 | 1480 | 520 |
| R9 | 2560 | 1480 | <40 | <40 | <40 | 90 | <40 | 1520 | 1560 | 2120 | 1520 |
| H1 | 10,240 | <40 | <40 | <40 | <40 | <40 | <40 | 90 | 6100 | 6270 | <40 |
| F78, R6, | 2120 | 95 | 95 | <40 | n.d. | 1520 | <40 | <40 | 2090 | 2120 | 1480 |
| R9, H1, | | | | | | | | | | | |
| N5 | | | | | | | | | | | |
| (SCPV) | | | | | | | | | | | |
| strain BK | n.a. | 7460 | 450 | <40 | 450 | <40 | <40 | <40 | <40 | <40 | <40 |
| HVR | | | | | | | | | | | |

TABLE 6B
Immunogenicity of Mimotope OMPC Conjugate Vaccine: ELISA by
Capture of Biotinylated Peptide, Reciprocal of GMT

| Mimotope- | | | | | | | | | | | |
|---|---|---|---|---|---|---|---|---|---|---|---|
| OMPC | | | | | | | | | | | |
| vaccine | H730 | H732 | H731 | 1012 | 1008 | 1009 | 1010 | 270 | 271 | 272 | 273 |
| F78 | 2120 | <40 | 1560 | 1520 | <40 | <40 | 380 | 130 | <40 | <40 | <40 |
| M63 | 8,490 | 5930 | 5,930 | 1,480 | 5,910 | 1,480 | 1480 | 2100 | 1480 | 1480 | 1480 |
| R6 | 640 | 390 | 530 | <40 | 1560 | <40 | <40 | <40 | <40 | <40 | <40 |
| R9 | 130 | 130 | 130 | 95 | 2120 | <40 | <40 | <40 | <40 | <40 | <40 |
| H1 | 95 | <40 | 95 | <40 | 2120 | <40 | <40 | <40 | <40 | <40 | <40 |
| F78, R6, | 2560 | 1520 | 2560 | 1520 | 1520 | <40 | 380 | 130 | <40 | 1480 | <40 |
| R9, H1, | | | | | | | | | | | |
| N5 | | | | | | | | | | | |
| (SCPV) | | | | | | | | | | | |
| Strain BK | 1810 | 1810 | <40 | <40 | <40 | <40 | 450 | <40 | <40 | <40 | 110 |
| HVR | | | | | | | | | | | |

TABLE 6C
Immunogenicity of Mimotope OMPC Conjugate Vaccine:
ELISA by Mab Capture of Recombinant Chimeric E2,
Reciprocal of GMT

| | F78-E2 | M63-E2 | R6-E2 | R9-E2 | H1-E2 | N5-E2 |
|---|---|---|---|---|---|---|
| F78 | 13,800 | <125 | 350 | <125 | <125 | 175 |
| M63 | 9,800 | 53,000 | 5,200 | <500 | 3,300 | <500 |
| R6 | 4,000 | 1730 | 16,000 | 3400 | 3300 | <125 |
| R9 | 800 | 645 | 800 | 9800 | 1290 | 350 |
| H1 | 1320 | 1000 | 1700 | 2400 | 4000 | <125 |
| F78, R6, R9, | 11,300 | 640 | 5200 | 9500 | 5200 | 38,000 |
| H1, N5 | | | | | | |
| (SCPV) | | | | | | |

The two most broadly reactive sera sets in these experiments were from the M63-conjugate and the SCPV consisting of five mimotope peptides. The anti-M63 sera recognized 5 of 10 peptides in the standard ELISA and 11 of 11 biotinylated peptides in the capture ELISA. The anti-five mimotope sera from SCPV animals recognized 6 of 10 peptides in the standard ELISA and 8 of 11 biotinylated peptides in the capture ELISA. Between these two sets all peptides are recognized with the exception of the 147-07 sequence. Also the reaction to the HVC H strain variant #2 is of low titer and should be grouped as a rare reactor. Thus, between a singular mimotope conjugate using M63 sequence and the SCPV, 19 of 21 HVR sequences were recognized.

Using chimeric E2 glycoproteins displaying each of the mimotopes, each set of immune sera strongly recognized E2 displaying its cognate mimotope (Table 6C) plus additional chimeric E2. The SCPV-elicited sera and the singular R9-elicited sera each recognized 6 of the 6 glycoproteins, while the singular F78 conjugate sera only bound to 3 of the 6 glycoproteins. The mimotope N5-chimera was the least reactive of the glycoproteins.

Two additional vaccine forms were prepared to use these mimotopes as immunogens in primate studies. First, the mimotope-E2 chimeric proteins were produced from mammalian cells, and secondly the DNA vectors expressing these same chimeric constructs were prepared. Each vaccine was tested in three rhesus monkeys, for a total of twelve vaccine studies in 36 animals. The immune sera were tested in gpELISA vs. the cognate chimeric glycoprotein and in all the peptide ELISAs using the cognate mimotope peptides and all the HVR peptides (Tables 7A and 7B).

TABLE 7A
Immunogenicity of Mimotope-E2 Chimeric Vaccine: Peptide ELISA,
Reciprocal of GMT

| Mimotope | Cognate | | HCV | | | | | | | | | |
|---|---|---|---|---|---|---|---|---|---|---|---|---|
| vaccine & | chimeric | cognate | BK | HCV H | HCV H | HCV H | HCV H | 147- | 147- | 147- | 147- | 147- |
| form | E2 | mimotope | HVR1 | HVR1 | HVR2 | HVR3 | HVR4 | 05 | 07 | 08 | 09 | 11 |
| F78 E2 | 4800 | 2980 | <40 | 200 | <40 | 95 | 1,490 | <40 | <40 | <40 | <40 | <40 |
| plasmid | | | | | | | | | | | | |
| vector | | | | | | | | | | | | |
| F78 E2 | 9800 | 6100 | <40 | 40 | <40 | <40 | 1480 | <40 | <40 | <40 | <40 | <40 |
| chimeric | | | | | | | | | | | | |
| protein | | | | | | | | | | | | |
| M63 E2 | 2000 | 1,560 | <40 | 90 | <40 | 90 | <40 | <40 | <40 | <40 | <40 | <40 |
| plasmid | | | | | | | | | | | | |
| vector | | | | | | | | | | | | |
| M63 E2 | 13,800 | 8490 | 370 | 360 | <40 | 360 | <40 | <40 | <40 | <40 | <40 | <40 |
| chimeric | | | | | | | | | | | | |
| protein | | | | | | | | | | | | |
| R6 E2 | 600 | 1,540 | <40 | <40 | <40 | <40 | <40 | <40 | <40 | <40 | <40 | <40 |
| plasmid | | | | | | | | | | | | |
| vector | | | | | | | | | | | | |
| R6 E2 | 4000 | 2560 | <40 | <40 | <40 | <40 | <40 | <40 | <40 | <40 | <40 | <40 |
| chimeric | | | | | | | | | | | | |
| protein | | | | | | | | | | | | |

TABLE 7B
Immunogenicity of Mimotope-E2 Chimeric Vaccine: Peptide ELISA,
Reciprocal of GMT

| | H730 | H732 | H731 | 1012 | 1008 | 1009 | 1010 | 270 | 271 | 272 | 273 |
|---|---|---|---|---|---|---|---|---|---|---|---|
| F78 E2 | 520 | <40 | 380 | <40 | <40 | <40 | 370 | 90 | <40 | <40 | <40 |
| plasmid | | | | | | | | | | | |
| vector | | | | | | | | | | | |
| F78 E2 | 90 | <40 | <40 | <40 | <40 | <40 | <40 | <40 | <40 | <40 | <40 |
| chimeric | | | | | | | | | | | |
| protein | | | | | | | | | | | |
| M63 E2 | 380 | 370 | 370 | <40 | <40 | <40 | <40 | <40 | <40 | <40 | <40 |
| plasmid | | | | | | | | | | | |
| vector | | | | | | | | | | | |
| M63 E2 | 370 | 370 | 370 | <40 | <40 | <40 | <40 | <40 | <40 | <40 | <40 |
| chimeric | | | | | | | | | | | |
| protein | | | | | | | | | | | |
| R6 E2 | <40 | <40 | <40 | <40 | <40 | <40 | <40 | <40 | <40 | <40 | <40 |
| plasmid | | | | | | | | | | | |
| vector | | | | | | | | | | | |
| R6 E2 | <40 | <40 | <40 | <40 | 90 | <40 | <40 | <40 | <40 | <40 | <40 |
| chimeric | | | | | | | | | | | |
| protein | | | | | | | | | | | |
| R9 E2 | <40 | <40 | <40 | <40 | <40 | <40 | <40 | <40 | <40 | <40 | <40 |
| plasmid | | | | | | | | | | | |
| vector | | | | | | | | | | | |
| R9 E2 | <40 | <40 | <40 | <40 | <40 | <40 | <40 | <40 | <40 | <40 | <40 |
| chimeric | | | | | | | | | | | |
| protein | | | | | | | | | | | |
| H1 E2 | <40 | <40 | <40 | <40 | <40 | <40 | <40 | <40 | <40 | <40 | <40 |
| plasmid | | | | | | | | | | | |
| vector | | | | | | | | | | | |
| H1 E2 | <40 | <40 | <40 | <40 | 95 | <40 | <40 | <40 | <40 | <40 | <40 |
| chimeric | | | | | | | | | | | |
| protein | | | | | | | | | | | |
| N5 E2 | <40 | <40 | <40 | <40 | <40 | <40 | <40 | <40 | <40 | <40 | <40 |
| plasmid | | | | | | | | | | | |
| vector | | | | | | | | | | | |
| N5 E2 | <40 | <40 | <40 | <40 | <40 | <40 | <40 | <40 | <40 | <40 | <40 |
| chimeric | | | | | | | | | | | |
| protein | | | | | | | | | | | |

All the immune sera recognized their cognate E2 glycoprotein, with the sera of the protein vaccinated animals consistently having a greater GMT than the DNA vector vaccinated animals. These anti-glycoprotein titers were also consistently greater than titers achieved against the mimotope peptides. In one case for R9-chimeric protein, no anti-mimotope response was observed, indicating that the ectodomain of the HCV E2 glycoprotein is not a particularly good delivery structure for eliciting broadly reactive anti-HVR antibodies.

Both forms of M63 and F78 immunogens elicited antibodies that recognized most of the H strain HVR sequences with GMT in the range of 1:40 to 1:1490, while only anti-M63 chimeric protein sera recognized the BK strain HVR peptide. Anti-H1 sera elicited by both forms of vaccine were able to recognize peptides # 147-08 and 147-09. This result was consistent with the observation that anti-H1 conjugate sera reacted strongly with these two sequences.

For the remainder of the peptides the entire body of sera did not react to any significant extent. These results suggest that DNA vectors of chimeric mimotope-E2 proteins and those mimotope-E2 proteins themselves are inferior anti-HVR immunogens. Taken together with results obtained with the mimotope conjugates, it appears that a correct set of HVR mimotopes properly presented as conjugate vaccines are capable of eliciting broadly reactive antibody responses and are superior to the other approaches.

Other embodiments are within the following claims. While several embodiments have been shown and described, various modifications may be made without departing from the spirit and scope of the present invention.

What is claimed is:

1. An HCV conjugate comprising:
a polypeptide or protein complex carrier,
immunogenic HCV peptide PEP₁,
immunogenic HCV peptide PEP₂,
wherein PEP₁ and PEP₂ are each covalently joined to said carrier though an independently selected covalent linker and comprises a different sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7; or a pharmaceutically acceptable salt thereof.

2. The conjugate of claim 1, further comprising immunogenic HCV peptide PEP₃, wherein PEP₃ is covalently joined to said carrier though an independently selected covalent linker and comprises a third sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7, wherein said third sequence comprises a different sequence than PEP₁ or PEP₂; or a pharmaceutically acceptable salt thereof.

3. The conjugate of claim 2, wherein PEP₁ comprises SEQ. ID. NO. 1, PEP₂ comprises SEQ. ID. NO. 2, and PEP₃ comprises SEQ. ID. NO. 4; or a pharmaceutically acceptable salt thereof.

4. The conjugate of claim 3, wherein said carrier is the Outer Membrane Protein Complex of Neisseria meningitidis.

5. The conjugate of claim 1, wherein said HCV conjugate comprises:
immunogenic HCV peptide PEP₁;
immunogenic HCV peptide PEP₂;
immunogenic HCV peptide PEP₃;
immunogenic HCV peptide PEP₄; and
immunogenic HCV peptide PEP₅;
wherein PEP₁, PEP₂, PEP₃, PEP₄, and PEP₅, are each covalently joined to said carrier though an independently selected covalent linker and comprises a different sequence selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7; or a pharmaceutically acceptable salt thereof.

6. The conjugate of claim 5, wherein said carrier is the Outer Membrane Protein Complex of Neisseria meningitidis.

7. The conjugate of claim 6, wherein PEP₁, PEP₂, PEP₃, PEP₄, and PEP₅ each consists essentially of a different sequence selected from the group consisting of: SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7; or a pharmaceutically acceptable salt thereof.

8. The conjugate of claim 1, wherein said conjugate has the following structure: 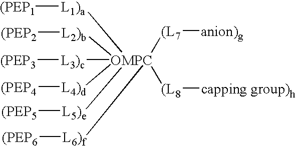
wherein OMPC represents the Outer Membrane Protein Complex of Neisseria meningitidis;
anion is a low molecular weight moiety having an anionic character at physiological pH;
L₁ is a covalent linker joining PEP₁ to OMPC, wherein each L₁ that is present may be the same or different;
L₂ is a covalent linker joining PEP₂ to OMPC, wherein each L₂ that is present may be the same or different;
L₃ is a covalent linker joining PEP₃ to OMPC, wherein each L₃ that is present may be the same or different;
L₄ is a covalent linker joining PEP₄ to OMPC, wherein each L₄ that is present may be the same or different;
L₅ is a covalent linker joining PEP₅ to OMPC, wherein each L₅ that is present may be the same or different;
L₆ is a covalent linker joining PEP₆ to OMPC, wherein each L₆ that is present may be the same or different;
L₇ is a covalent linker joining anion to OMPC, wherein each L₇ and each anion that are present may be the same or different;
L₈ is a covalent linker joining capping group to OMPC, wherein each L₈ and each capping group that is present may be the same or different;
a, b, c, d, e, f, g, and h is each an individually selected coupling load, wherein a, b, c, and h are each greater than 0;
PEP₁, PEP₂, PEP₃, PEP₄, PEP₅, and PEP₆, are immunogenic peptides, provided that at least two of PEP₁, PEP₂, PEP₃, PEP₄, PEP₅, and PEP₆ comprise a sequence selected from the group consisting of SEQ. I). NO. 1, SEQ. ID. NO. 2, SEQ. ID. NO. 3, SEQ. ID. NO. 4, SEQ. ID. NO. 5, and SEQ. ID. NO. 6;
or a pharmaceutically acceptable salt thereof.

9. The conjugate of claim 8, wherein
each of L₁, L₂, L₃, L₄, L_{5, 4}, L₇, and L₈ independently has the structure: 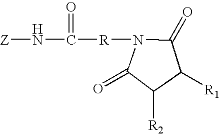
wherein OMPC is joined at position “z”,
R is selected from the group consisting of alkylene, substituted alkylene, and phenyl;
one of R₁ and R₂ is either hydrogen, alkyl, substituted alkyl or —SO₃H, and the other of R₁ and R₂ is the position to which PEP₁, PEP₂, PEP₃, PEP₄, PEP₅, PEP₆, anion, or capping group is joined; and
each anion is either carboxylic, sulfonic, propionic, or phosphonic acid;
or a pharmaceutically acceptable salt thereof.

10. The conjugate of claim 9, wherein g is zero and each of L₁, L₂, L₃, L₄, L₅, L₆, and L₈ have the following structure: 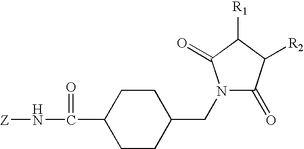
wherein OMPC is joined at position “z”,
one of R₁ and R₂ is hydrogen, and the other of R₁ and R₂ is the position to which PEP₁, PEP₂, PEP₃, PEP₄, PEP₅, PEP₆, or capping group join; and
each of PEP₁, PEP₂, PEP₃, PEP₄, PEP₅, and PEP₆ consists of a different sequence selected from the group consisting of SEQ. ID. NO. 8, SEQ. ID. NO. 9, SEQ. ID. NO. 10, SEQ. ID. NO. 11, SEQ. ID. NO. 12, SEQ. ID. NO. 13, and SEQ. ID. NO. 14;
or a pharmaceutically acceptable salt thereof.

11. The conjugate of claim 10, wherein each of PEP₁, PEP₂, PEP₃, PEP₄, PEP₅, and PEP₆ consists of a different sequence selected from the group consisting of SEQ. ID. NO. 15, SEQ. ID. NO. 16, SEQ. ID. NO. 17, SEQ. ID. NO. 18, SEQ. ID. NO. 19, SEQ. ID. NO. 20, and SEQ. ID. NO. 21; or a pharmaceutically acceptable salt thereof.

12. The conjugate of claim 10, wherein f is zero,
PEP₁ consists of SEQ. ID. NO. 15;
PEP₂ consists of SEQ. ID. NO. 16;
PEP₃ consists of SEQ. ID. NO. 18;
or a pharmaceutically acceptable salt thereof.

13. The conjugate of claim 12, wherein d is greater than zero, e is greater than zero, PEP₄ consists of SEQ. ID. NO. 17, and PEP₅ consists of SEQ. ID. NO. 19; or a pharmaceutically acceptable salt thereof.

14. The conjugate of claim 10, wherein d is greater than zero, e is greater than zero, f is zero, PEP₁ consists of SEQ. ID. NO. 15, PEP₂ consists of SEQ. ID. NO. 17, PEP₃ consists of SEQ. ID. NO. 18, PEP₄ consists of SEQ. ID. NO. 19, and PEP₅ consists of SEQ. ID. NO. 20; or a pharmaceutically acceptable salt thereof.

15. The conjugate of claim 13, wherein each of said capping group is N-acetylcysteine.

16. The conjugate of claim 14, wherein each of said capping group is N-acetylcysteine.

17. An HCV conjugate mixture comprising a first and a second different HCV conjugate wherein,
said first HCV conjugate is the conjugate of any one of claims 1-16; and
said second conjugate comprises a second polypeptide or protein carrier covalently joined to an immunogenic HCV peptide comprising SEQ. ID. NO. 7; or a pharmaceutically acceptable salt thereof.

18. The conjugate mixture of claim 17, said second carrier is the Outer Membrane Protein Complex of Neisseria meningitidis.

19. The conjugate of mixture of claim 18, wherein said second conjugate comprises an immunogenic HCV peptide consisting essentially of the sequence of SEQ. ID. NO. 7; or a pharmaceutically acceptable salt thereof.

20. The conjugate mixture of claim 19, wherein said second conjugate has the following structure: 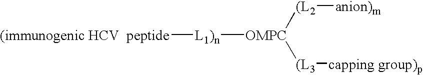
wherein OMPC represents the Outer Membrane Protein Complex of Neisseria meningitidis;
each anion is a low molecular weight moiety having an anionic character at physiological pH;
L₁ is a covalent linker joining the immunogenic HCV peptide to OMPC, wherein each L₁ may be the same or different;
L₂ is a covalent linker joining anion to OMPC, wherein each L₂ and anion that is present may be the same or different;
L₃ is a covalent linker joining the capping group to OMPC, wherein each L₃ and each capping group that is present may be the same or different;
n, m, and p is each an individually selected coupling load, wherein n and p are each greater than 0;
or a pharmaceutically acceptable salt thereof.

21. The conjugate mixture of claim 20, wherein for said second conjugate each of L₁, L₂ and L₃ independently have the following structure: 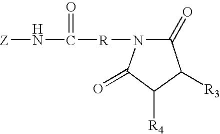
wherein OMPC is joined at position “z”,
R is selected from the group consisting of alkylene, substituted alkylene, and phenyl;
one of R₃ and R₄ is either hydrogen, alkyl, substituted alkyl or —SO₃H, and the other of R₃ and R₄ is the position to which the immunogenic peptide, anion, or capping group binds;
each anion is either carboxylic, sulfonic, propionic, or phosphonic acid; and
said immunogenic HCV peptide consists of the sequence of SEQ. ID. NO. 14;
or a pharmaceutically acceptable salt thereof.

22. The conjugate mixture of claim 21, wherein for said second conjugate m is zero and L₁ and L₃ has the following structure: 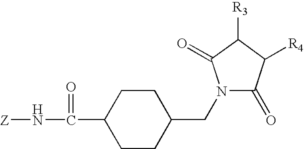
wherein OMPC is joined at position “z”,
one of R₃ and R₄ is hydrogen, and the other of R₃ and R₄ is the position to which said immunogenic peptide or capping group binds.

23. The conjugate mixture of claim 22, wherein said second conjugate immunogenic peptide consists of SEQ. ID. NO. 21 or a pharmaceutically acceptable salt thereof.

24. The conjugate mixture of claim 23, wherein each of said capping group in said second conjugate is N-acetylcysteine.

25. An HCV conjugate produced by process comprising:
a) joining a plurality of linkers to reactive sites on a polypeptide or protein complex carrier;
b) joining two or more different HCV immunogenic peptides to said plurality of linkers; and
c) capping the product of step (b);
wherein each of said two or more different HCV immunogenic peptides is selected from the group consisting of: a first HCV mimotope sequence comprising SEQ. ID. NO. 1; a second HCV mimotope sequence comprising SEQ. ID. NO. 2; a third HCV mimotope sequence comprising SEQ. ID. NO. 3; a fourth HCV mimotope sequence comprising SEQ. ID. NO. 4; a fifth HCV mimotope sequence comprising SEQ. ID. NO. 5; a sixth HCV mimotope sequence comprising SEQ. ID. NO. 6; and a seventh HCV mimotope sequence comprising SEQ. ID. NO. 7.

26. The HCV conjugate of claim 25, wherein said step (b) is performed by simultaneous conjugation of said two or more different HCV immunogenic peptides.

27. The HCV conjugate of claim 26, wherein said two or more different HCV immunogenic peptides comprise:
a first HCV immunogenic peptide comprising said first HCV mimotope sequence,
a second HCV immunogenic peptide comprising said second HCV mimotope sequence, and
a third HCV immunogenic peptide comprising said third HCV mimotope sequence.

28. A method of inducing an immune response comprising the step of inoculating a subject with an effective amount of the conjugate of claim 1.

29. The method of claim 28, wherein said subject is a human.

30. A method of inducing an immune response comprising the step of inoculating a subject with an effective amount of the conjugate mixture of claim 17.

31. The method of claim 30, wherein said subject is a human.

32. A method of making the conjugate of claim 1, comprising the step of simultaneously conjugating PEP₁ and PEP₂ to said carrier.

33. A method of making the conjugate of claim 4, comprising the step of simultaneously conjugating PEP₁, PEP₂, PEP₃, and PEP₄ to said carrier.

34. An antisera made by a process comprising the steps of:
a) inoculating a subject with an effective amount of the conjugate of claim 1 to produce antibodies; and
b) removing said antibodies from said subject.

35. An antisera made by a process comprising the steps of:
a) inoculating a subject with an effective amount of the conjugate mixture of claim 17 to produce antibodies; and
b) removing said antibodies from said subject.

36. An HCV conjugate comprising:
a polypeptide or protein complex carrier; and
an immunogenic HCV peptide comprising an HCV mimotope selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, and SEQ. ID. NO. 7; wherein said immunogenic HCV peptide is covalently joined to said carrier;
wherein said carrier is the Outer Membrane Protein Complex of Neisseria meningitidis;
or a pharmaceutically acceptable salt thereof.

37. The conjugate of claim 36, wherein
said HCV mimotope comprises the sequence of SEQ. ID. NO. 7;
or a pharmaceutically acceptable salt thereof.

38. The conjugate of claim 37, wherein said immunogenic HCV peptide consists essentially of the sequence of SEQ. ID. NO. 7 or a pharmaceutically acceptable salt thereof.

39. The conjugate of claim 36, wherein said conjugate has the following structure: 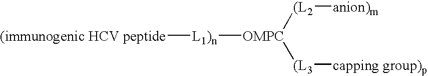
wherein OMPC represents the Outer Membrane Protein Complex of Neisseria meningitidis;
each anion is a low molecular weight moiety having an anionic character at physiological pH;
L₁ is a covalent linker joining the immunogenic HCV peptide to OMPC, wherein each L₁ may be the same or different;
L₂ is a covalent linker joining anion to OMPC, wherein each L₂ and anion that is present may be the same or different;
L₃ is a covalent linker joining the capping group to OMPC, wherein each L₃ and each capping group that is present may be the same or different;
n, m, and p is each an individually selected coupling load, wherein n and p are each greater than 0;
or a pharmaceutically acceptable salt thereof.

40. The conjugate of claim 39, wherein each of L₁, L₂ and L₃ independently have the following structure: 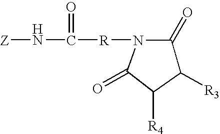
wherein OMPC is joined at position “z”,
R is selected from the group consisting of alkylene, substituted alkylene, and phenyl;
one of R₃ and R₄ is either hydrogen, alkyl, substituted alkyl or —SO₃H, and the other of R₃ and R₄ is the position to which the immunogenic peptide, anion, or capping group binds;
each anion is either carboxylic, sulfonic, propionic, or phosphonic acid; and
said immunogenic HCV peptide consists of the sequence of SEQ. ID. NO. 14;
or a pharmaceutically acceptable salt thereof.

41. The conjugate of claim 40, wherein m is zero and L₁ and L₃ has the following structure: 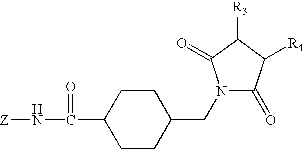
wherein OMPC is joined at position “z”,
one of R₃ and R₄ is hydrogen, and the other of R₃ and R₄ is the position to which said immunogenic peptide or capping group binds.

42. The conjugate of claim 41, wherein said immunogenic peptide consists of SEQ. ID. NO. 21 or a pharmaceutically acceptable salt thereof.

43. The conjugate of claim 40, wherein each of said capping group is N-acetylcysteine.

44. A method of inducing an immune response comprising the step of inoculating a subject with an effective amount of the conjugate of claim 36.

45. The method of claim 44, wherein said subject is a human.

46. An antisera made by a process comprising the steps of:
a) inoculating a subject with an effective amount of the conjugate claim 36 to produce antibodies; and
b) removing said antibodies from said subject.


SEQUENCE LISTING (52 sequences)

SEQ ID NO: 1 - protein, 27 aa, Artificial Sequence
  MOD_RES (1)...(1): Xaa = Glutamine or pyroglutamate
XTHTTGGQAGHQAHSLTGLFSPGAKQN

SEQ ID NO: 2 - protein, 27 aa, Artificial Sequence
  MOD_RES (1)...(1): Xaa = Glutamine or pyroglutamate
XTTTTGGQVSHATHGLTGLFSLGPQQK

SEQ ID NO: 3 - protein, 27 aa, Artificial Sequence
  MOD_RES (1)...(1): Xaa = Glutamine or pyroglutamate
XTTVVGGSQSHTVRGLTSLFSPGASQN

SEQ ID NO: 4 - protein, 27 aa, Artificial Sequence
  MOD_RES (1)...(1): Xaa = Glutamine or pyroglutamate
XTHTTGGVVGHATSGLTSLFSPGPSQK

SEQ ID NO: 5 - protein, 27 aa, Artificial Sequence
  HCV mimotope
TTTTTGGQVGHQTSGLTGLFSPGAQQN

SEQ ID NO: 6 - protein, 27 aa, Artificial Sequence
  HCV mimotope
TTTTTGGVQGHTTRGLVRLFSLGSKQN

SEQ ID NO: 7 - protein, 27 aa, Artificial Sequence
  MOD_RES (1)...(1): Xaa = Glutamine or pyroglutamate
XTHTTGGVVSHQTRSLVGLFSPGPQQN

SEQ ID NO: 8 - protein, 29 aa, Artificial Sequence
  MOD_RES (1)...(1): Xaa = Glutamine or pyroglutamate
XTHTTGGQAGHQAHSLTGLFSPGAKQNXX

SEQ ID NO: 9 - protein, 29 aa, Artificial Sequence
  MOD_RES (1)...(1): Xaa = Glutamine or pyroglutamate
XTTTTGGQVSHATHGLTGLFSLGPQQKXX

SEQ ID NO: 10 - protein, 29 aa, Artificial Sequence
  MOD_RES (1)...(1): Xaa = Glutamine or pyroglutamate
XTTVVGGSQSHTVRGLTSLFSPGASQNXX

SEQ ID NO: 11 - protein, 29 aa, Artificial Sequence
  MOD_RES (1)...(1): Xaa = Glutamine or pyroglutamate
XTHTTGGVVGHATSGLTSLFSPGPSQKXX

SEQ ID NO: 12 - protein, 29 aa, Artificial Sequence
  HCV mimotope
TTTTTGGQVGHQTSGLTGLFSPGAQQNXX

SEQ ID NO: 13 - protein, 29 aa, Artificial Sequence
  HCV mimotope
TTTTTGGVQGHTTRGLVRLFSLGSKQNXX

SEQ ID NO: 14 - protein, 29 aa, Artificial Sequence
  MOD_RES (1)...(1): Xaa = Glutamine or pyroglutamate
XTHTTGGVVSHQTRSLVGLFSPGPQQNXX

SEQ ID NO: 15 - protein, 29 aa, Artificial Sequence
  MOD_RES (1)...(1): Xaa = Glutamine or pyroglutamate
XTHTTGGQAGHQAHSLTGLFSPGAKQNXC

SEQ ID NO: 16 - protein, 29 aa, Artificial Sequence
  MOD_RES (1)...(1): Xaa = Glutamine or pyroglutamate
XTTTTGGQVSHATHGLTGLFSLGPQQKXC

SEQ ID NO: 17 - protein, 29 aa, Artificial Sequence
  MOD_RES (1)...(1): Xaa = Glutamine or pyroglutamate
XTTVVGGSQSHTVRGLTSLFSPGASQNXC

SEQ ID NO: 18 - protein, 29 aa, Artificial Sequence
  MOD_RES (1)...(1): Xaa = Glutamine or pyroglutamate
XTHTTGGVVGHATSGLTSLFSPGPSQKXC

SEQ ID NO: 19 - protein, 29 aa, Artificial Sequence
  MOD_RES (28)...(28): Xaa = Norvaline, norleucine, 2-aminobutyric acid, 2,4-diaminobutyric acid, 2,3 diaminopropionic acid, or cyclohexylglycine
TTTTTGGQVGHQTSGLTGLFSPGAQQNXC

SEQ ID NO: 20 - protein, 29 aa, Artificial Sequence
  MOD_RES (28)...(28): Xaa = Norvaline, norleucine, 2-aminobutyric acid, 2,4-diaminobutyric acid, 2,3 diaminopropionic acid, or cyclohexylglycine
TTTTTGGVQGHTTRGLVRLFSLGSKQNXC

SEQ ID NO: 21 - protein, 29 aa, Artificial Sequence
  MOD_RES (1)...(1): Xaa = Glutamine or pyroglutamate
XTHTTGGVVSHQTRSLVGLFSPGPQQNXC

SEQ ID NO: 22 - protein, 56 aa, Artificial Sequence
  MOD_RES (1)...(1): Xaa = Glutamine or pyroglutamate
XTHTTGGQAGHQAHSLTGLFSPGAKQNQTHTTGGVVSHQTRSLVGLFSPGPQQNXX

SEQ ID NO: 23 - protein, 56 aa, Artificial Sequence
  MOD_RES (1)...(1): Xaa = Glutamine or pyroglutamate
XTHTTGGQAGHQAHSLTGLFSPGAKQNQTHTTGGVVSHQTRSLVGLFSPGPQQNXC

SEQ ID NO: 24 - protein, 29 aa, Artificial Sequence
  MOD_RES (1)...(1): Xaa = Glutamine or pyroglutamate
XTHTTGGQAGHQAHSLTGLFSPGAKQNXC

SEQ ID NO: 25 - protein, 29 aa, Artificial Sequence
  MOD_RES (1)...(1): Xaa = Glutamine or pyroglutamate
XTHTTGGQAGHQAHSLTGLFSPGAKQNXC

SEQ ID NO: 26 - protein, 29 aa, Artificial Sequence
  MOD_RES (1)...(1): Xaa = Glutamine or pyroglutamate
XTTTTGGQVSHATHGLTGLFSLGPQQKXC

SEQ ID NO: 27 - protein, 29 aa, Artificial Sequence
  MOD_RES (1)...(1): Xaa = Glutamine or pyroglutamate
XTTVVGGSQSHTVRGLTSLFSPGASQNXC

SEQ ID NO: 28 - protein, 29 aa, Artificial Sequence
  MOD_RES (1)...(1): Xaa = Glutamine or pyroglutamate
XTHTTGGVVGHATSGLTSLFSPGPSQKXC

SEQ ID NO: 29 - protein, 29 aa, Artificial Sequence
  MOD_RES (28)...(28): Xaa = 2,4-Diaminobutyric acid
TTTTTGGQVGHQTSGLTGLFSPGAQQNXC

SEQ ID NO: 30 - protein, 29 aa, Artificial Sequence
  MOD_RES (28)...(28): Xaa = Norleucine
TTTTTGGVQGHTTRGLVRLFSLGSKQNXC

SEQ ID NO: 31 - protein, 29 aa, Artificial Sequence
  MOD_RES (1)...(1): Xaa = Glutamine or pyroglutamate
XTHTTGGVVSHQTRSLVGLFSPGPQQNXC

SEQ ID NO: 32 - protein, 27 aa, Artificial Sequence
  HVR peptide
ETHVTGGNAGRTTAGLVGLLTPGAKQN

SEQ ID NO: 33 - protein, 27 aa, Artificial Sequence
  HVR peptide
ETHVTGGSAGRTIAGFTGLLTPGAKQN

SEQ ID NO: 34 - protein, 27 aa, Artificial Sequence
  HVR peptide
ETHVTGGSAGHTTAGLVGLLTPGAKQN

SEQ ID NO: 35 - protein, 27 aa, Artificial Sequence
  HVR peptide
ETHVTGGSAGHTTAGLVRLLSPGAKQN

SEQ ID NO: 36 - protein, 27 aa, Artificial Sequence
  HVR peptide
NTRVTGGVQSHTTRGFVGMFSLGPSQR

SEQ ID NO: 37 - protein, 27 aa, Artificial Sequence
  HVR peptide
ETHVTGGSAAYAAHGLANILSRGAKQD

SEQ ID NO: 38 - protein, 27 aa, Artificial Sequence
  HVR peptide
TTYTTGGSAGHSLPGISNLFAPGAQQH

SEQ ID NO: 39 - protein, 27 aa, Artificial Sequence
  HVR peptide
DTYTTGGSAARSTLGLTSLFVAGPKQN

SEQ ID NO: 40 - protein, 27 aa, Artificial Sequence
  HVR peptide
FTRVTGGAQAVPTHGLTSLFTFGAQQN

SEQ ID NO: 41 - protein, 27 aa, Artificial Sequence
  HVR peptide
NTYVTGGQAGYTTMALSSLFAPGAQQN

SEQ ID NO: 42 - protein, 27 aa, Artificial Sequence
  HVR peptide
ETHVTGGNAGRTTAGLVGLLTPGAKQN

SEQ ID NO: 43 - protein, 27 aa, Artificial Sequence
  HVR peptide
ETHVTGGNAGRTTAGLVGLLTPDAKQN

SEQ ID NO: 44 - protein, 27 aa, Artificial Sequence
  HVR peptide
ETHVTGGNAGRTAAGLVGLLTPGAKQN

SEQ ID NO: 45 - protein, 27 aa, Artificial Sequence
  HVR peptide
ETHVTGGSAGHTVSGFVSLLAPGAKQN

SEQ ID NO: 46 - protein, 27 aa, Artificial Sequence
  HVR peptide
NTYVTGGAAARGASGITSLFSRGPSQK

SEQ ID NO: 47 - protein, 27 aa, Artificial Sequence
  HVR peptide
GTTRVGGAAARTTSSFASLLTHGPSQN

SEQ ID NO: 48 - protein, 27 aa, Artificial Sequence
  HVR peptide
ETRVTGGAAGHTAFGFASFLAPGAKQN

SEQ ID NO: 49 - protein, 27 aa, Artificial Sequence
  HVR peptide
DTHVVGGATERTAYSLTGLFTAGPKQN

SEQ ID NO: 50 - protein, 27 aa, Artificial Sequence
  HVR peptide
GTTCQGGVYARGAGGIASLFSVGANQK

SEQ ID NO: 51 - protein, 27 aa, Artificial Sequence
  HVR peptide
RTLSFGGLPGHTTHGFASLSAPGAKQN

SEQ ID NO: 52 - protein, 27 aa, Artificial Sequence
  HVR peptide
RTILMAGRQAEVTQSFPGLFSLAPSQK